(19) United States
(12) Patent Application Publication
(10) Pub. No.: US 2008/0241082 A1
(43) Pub. Date: Oct. 2, 2008

(54) Method for the Preparation of Cosmetic Emulsion

(75) Inventors: Jacob Guth, Upper Black Eddy, PA (US); Vickie Lentner, Hunterdon, NJ (US)

Correspondence Address: FOLEY AND LARDNER LLP;SUITE 500, 3000 K STREET NW, WASHINGTON, DC, 20007, US

(73) Assignee: Lonza Inc., Allendale, NJ (US)

(21) Appl. No.: 10/596,116

(22) PCT Filed: Apr. 4, 2005
(86) PCT No.: PCT/EP05/03501

§ 371 Date: Mar. 4, 2008

Related U.S. Application Data
(60) Provisional application No. 60/559,832, filed on Apr. 5, 2004.

(30) Foreign Application Priority Data
Jul. 8, 2004 (EP) 04016057.4

(51) Int. Cl. A61K 8/06; A61K 8/97; A61Q 1/10; A61Q 17/04

(52) U.S. Cl. 424/59; 424/63

(57) ABSTRACT

A novel method is disclosed for the preparation of oil-in-water emulsions suitable for cosmetic or topical dermatological products. The method comprises the step of mixing at least one cosmetically active ingredient with an aqueous emulsion of washed vegetable oil bodies (oleosomes) and the temperature of the products does never exceed about 40° C. during the steps of the method.

### FIELD OF THE INVENTION

This invention relates to a method for the preparation of oil-in-water emulsions suitable for cosmetic or topical dermatological products.

### BACKGROUND OF THE INVENTION

Oil-in-water (O/W) emulsions typically contain between 5 and 40% by weight of oil and/or oil soluble (hydrophobic) ingredients and between 60 and 95% by weight of water and/or water soluble (hydrophilic) ingredients.

O/W emulsions are normally prepared by combining the oil phase and the water phase together at high temperature (80-85° C.) and with vigorous agitation in the presence of an emulsifier until the oil droplets are completely dispersed within the water. The emulsifying agent is generally part of the oil phase, and is present at a level of between 10 and 30% by weight of the total oil phase. The resulting emulsion must then be continuously agitated during the cooling process in order to prevent separation (of the emulsion) into the original two phases. Typically agitation is required until the emulsion has cooled to a temperature of 40° C. or below. The process of heating the oil and water phases in order to achieve the emulsion is very time and energy consuming as is the cooling down phase. Moreover, in products for topical application the presence of the emulsifier in the finished emulsion presents a number of drawbacks:
1) The presence of the emulsifier reduces the water repellency of the formulation once it has been applied to the skin. This is particularly important for sunscreen applications and color cosmetics.
2) In skin care products, the presence of the emulsifier increases the irritancy potential of the formulation, in that it has a delipidizing effect on the skin.

Vegetable oil bodies (oleosomes) or oil microspheres are discrete spheres which occur naturally in the seeds of oilseed crops. They consist of an outer coat of phospholipids and proteins (oleosins) and an internal liquid, semi-solid, or low melting solid collection of the tri-glycerides associated with the individual plant seed. For example, safflower oil microspheres (oleosomes) would contain the triglycerides with the fatty acid content normally associated with that plant seed, i.e., 6.5% palmitic, 2.5% stearic, 11.5% oleic, 79% linoleic, and 0.5% linolenic acids. Size and properties of these microspheres are dependent on the seed type, but for safflower seeds the microspheres are typically 3 μm in diameter and can be obtained as a 70-75 wt. % emulsion in water.

The preparation and several applications of oil bodies (oleosomes) are described in a number of patents assigned to the Canadian company Sembiosys Genetics Inc., e.g. U.S. Pat. No. 6,146,645. Three of these patents are discussed in the following.

Deckers et al., U.S. Pat. No. 6,183,762, disclose the preparation of cosmetic compositions for various applications. However, in each one of the applications described, the oil and water phases of the emulsion are heated to temperatures of 70° C. or higher whereupon they are added together with agitation. After the emulsion has been prepared and cooling has taken place (to 40° C. or lower ) then the oil body (microsphere) emulsion is added.

In U.S. Pat. No. 6,582,710, the same inventors claim the use of vegetable oil microspheres (oleosomes) in cosmetic applications. Again all of the formulations are prepared in the traditional way, i.e. through the process of heating the oil and water phases independently, combining them together with agitation, and then cooling to 40° C. before addition of the oleosomes.

Another patent by the same inventors, U.S. Pat. No. 6,599,513, describes the use of oil bodies to achieve finished formulations applicable to cosmetics. Again all of the Examples comprise a heating step.

### SUMMARY OF THE INVENTION

We have found that we can prepare stable, cosmetically acceptable emulsions simply by adding the requisite water or oil soluble ingredients directly to the dispersion of washed oil bodies (oleosomes) with stirring. No lengthy heating period is required and obviously no cooling period is needed either; the process comprises no heating step at all. The advantages of such an approach in terms of energy savings, preparation time, and in increased product throughput should be obvious.

### DETAILED DESCRIPTION OF THE INVENTION

As hereinbefore mentioned, the cosmetic or topical dermatological emulsions are prepared according to the present invention by adding the cosmetically or dermatologically active ingredients and, optionally, water to a dispersion of washed oil bodies (oleosomes) under stirring without any heating, at a temperature never exceeding about 40° C., to obtain an oil-in-water emulsion containing at least 60% by weight of water. The order of addition is not critical and the ingredients may be added one at a time or in premixed form, e.g. water-soluble components as aqueous solution or mutually miscible liquid ingredients as a mixture. Additional oil soluble ingredients (e.g. UV chromophores, vitamins, etc.) as well as other adjuvants and additives can also be added to the dispersion of oil bodies. The stirring can be effected by essentially any sort of stirrer or impeller operated at appropriate speed.

The cosmetic or topical dermatological products that can be prepared by the method of the present invention are essentially all products comprising oil-in-water emulsions in lotion, cream or paste form, including, but not limited to, moisturizing preparations, ointments, skin reparatives, sunscreens, skin lighteners, and decorative cosmetics such as color foundation creams, eye shadow creams and tanner creams.

Accordingly, they may comprise ingredients designed to exfoliate, repair, color, lighten, moisturize, smooth, condition, protect, prevent the moisture loss of, reverse the damage of, and clean and rejuvenate the skin and hair.

Such ingredients may include but are not limited to alcohols, esters, fatty acids, hydrocarbons, silicones, amines, enzymes, salts of amines and salts of fatty acids.

Other ingredients which could be added would include thickeners, barrier agents, UV chromophores, emollients, emulsifiers, conditioners, vitamins, etc.

Preferably, the emulsion is a cosmetic product is selected from the group consisting of moisturizers, ointments, cleansers, makeup removers, toners, night treatments, skin reparatives, sunscreens, skin lighteners, color foundations, eye shadows and tanners.

In one preferred embodiment, the product is a lotion.

In another preferred embodiment, the product is a cream.

The vegetable oil bodies (oleosomes) can be obtained from any plant cells, including cells from pollens, spores, seeds and vegetative plant organs in which oil bodies or oil body-like organelles are present. Preferably, the vegetable oil bodies are obtained from plant seeds or fruits, more preferably from the group of plant species consisting of rapeseed, soybean, sunflower, oil palm, cottonseed, peanut, walnut, coconut, castor, safflower, mustard, coriander, squash, linseed, brazil nut, jojoba, avocado and maize. Most preferably, the vegetable oil bodies are obtained from safflower seeds.

The preparation of the vegetable oil bodies (oleosomes) is disclosed in several patents, for example, U.S. Pat. No. 6,146,645. The preparation process comprises at least one washing step to accomplish removal of contaminating seed material, which is essential for obtaining a dispersion having properties suitable for being used in cosmetics. Furthermore, said removal of contaminating material reduces the risk of allergic reactions when the cosmetic preparation is applied to the skin.

The method of the present invention requires no additional emollients, emulsifiers etc. which constitutes a substantial advantage over the methods and products of prior art. In a preferred embodiment, no additional emulsifier is used.

The oleosomes are preferably used in an amount of from 2 wt. % to 15 wt. %, more preferably 5 wt. % to 12 wt. %, of a 70-75 wt. % emulsion in water, based on the total weight of the cosmetic or topical dermatological product.

The cosmetic or topical dermatological product produced according to the method of the present invention are remarkably stable and do not show any phase separation even after several months at elevated temperatures of e.g. 45° C.

The following non-limiting examples are illustrative of the present invention:

### EXAMPLES

### General Procedure:

All preparations have been carried out using a 2 L beaker and a Heidolph® RZR50 stirrer equipped with an anchor-type impeller of 10 cm diameter The stirring speed is given in min⁻¹ (1 min⁻=1 revolution per minute≡1 rpm). The batch size was 1 kg in each example.

### Materials:

All materials used in the examples are commercially available, in particular:

Stearic acid, octyl methoxycinnamate, paraffin oil, cetyl alcohol, urea, vitamin A, vitamin E, glycerol, arbutin, polysorbate 80 and hydroxyethylcellulose are standard chemicals obtainable from several suppliers. Decaglycerol L DE (Natrulon® H 10), L-carnitine, Geogard® 361, Lonzest® 143S (myristyl propionate), sorbitan monooleate and Aldo® MCT (medium chain triglycerides) are supplied by Lonza Inc., Allendale, N.J., USA. Novemer® EC-1 is available from Noveon Inc., Cleveland, Ohio, USA. Almond fragrance 901A79 is available from Flavor & Fragrance Specialties Inc., Mahwah, N.J., USA. Safflower (Carthamus tinctorius L.) oil microspheres (oleosomes) are available from Sembiosys Genetics Inc., Calgary, Canada. Kelzan®-T is available from CP Kelco, Wilmington, Del., USA. The pigments used in examples 8 to 13, i.e., green color pigment, red cosmetic iron oxide, brown iron oxide and titanium dioxide are supplied by Whittaker, Clark & Daniels Inc., South Plainfield, N.J., USA. Papain is available from Enzybel S. A., Villers-le-Bouillet, Belgium and bromelain from Marcor Development Corp., Carlstadt, N.J., USA. Rumex occidentalis extract is obtainable from Atrium Biotechnologies Inc., Fairfield, N.J., USA.

An example of a typical sunscreen formulation prepared in the traditional way is shown below in Comparative Example 1. The process for preparation of a similar sunscreen via a “cold process” approach is shown in Example 1. Examples 2 to 15 relate to various cosmetic formulations.

### Comparative Example 1

Sunscreen (SPF ≅ 13)

| Ingredients | % Weight |
|---|---|
| Stearic Acid | 6.00 |
| Octyl Methoxycinnamate | 8.00 |
| Paraffin Oil | 3.00 |
| Cetyl Alcohol | 2.00 |
| Myristyl Propionate (Lonzest ® 143S) | 3.00 |
| Medium Chain Triglycerides (Aldo ® MCT) | 3.00 |
| Sorbitan Monooleate | 5.05 |
| Polysorbate 80 | 3.95 |
| Water | 65.60 |
| Geogard ® 361 (Preservative) | 0.40 |
| Novemer ® EC-1 | 1.00 |
| Almond Fragrance # 901A79 | q.s. |

SPF = sun protection factor,
q.s. = quantum sufficit,
“≅” = approximately

### Procedure

Stearic acid, octyl methoxycinnamate, paraffin oil, cetyl alcohol, myristyl propionate, medium chain triglycerides, sorbitan monooleate and polysorbate 80 are mixed (phase 1) and heated to 80-85° C. with stirring to avoid charring and overheating. Water (phase 2) is heated to 80-85° C. Phase 1 is added to phase 2 with vigorous string over a 30 minute time period. The temperature is maintained at 80° C. for an additional 30 min while agitation continues. The emulsion is then cooled while stirring vigorously until a temperature of 40° C. or less is achieved, whereupon the preservative, Novemer® and the fragrance is added. Total process time is approximately 4.5 hours.

### Example 1

Sunscreen (SPF ≅ 15)

| Ingredients | % Weight |
|---|---|
| Octyl Methoxycinnamate | 8.00 |
| Water | 76.60 |
| Safflower Oleosomes (70%) | 8.00 |
| Decaglycerol L DE (Natrulon ® H 10) | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes and mix at 500 min⁻¹ for 10 min. Add the decaglycerol to the mixture with stirring for 2 min at 500 min⁻¹. Add the methoxycinnamate and the preservative to the mixture and stir at 500 min^{'1} for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 10,240 mPa·s The formulation passed stability testing at 45° C. for two months.

Thus the formulation of Example 1 in which safflower oleosomes were substituted for the diluents, emollients and emulsifiers that were used in the Comparative Example 1 was prepared with no heating, no cooling, in much less time, and with far fewer ingredients.

### Example 2

Emulsifier Free Sunscreen

| Ingredients | % Weight |
|---|---|
| Octyl Methoxycinnamate | 8.00 |
| Water | 76.60 |
| Safflower Oleosomes (70%) | 8.00 |
| Decaglycerol L DE (Natrulon ® H 10) | 5.00 |
| Hydroxyethylcellulose (Natrosol ®) | 2.00 |
| Geogard ® 361 (Preservative) | 0.40 |
| Almond Fragrance # 901A79 | q.s. |

### Procedure

Add the water to the safflower oleosomes and mix at 500 min⁻¹ for 5 min. Add the decaglycerol to the mixture with stirring for 2 min at 500 min⁻¹. Add the methoxycinnamate and the preservative to the mixture and stir at 500 min⁻¹ for 8 min. Add Natrosol® (presolubilized) and stir at 500 min⁻¹ until the mixture begins to thicken and then increase the stirrer speed to 600 min⁻¹ and stir for an additional 15 min. Total preparation time is ˜30 min, viscosity 22,800 mPa·s. The formulation passed stability testing at 40° C. for two months.

Many other cosmetic or topical dermatological products can be prepared in stable form via the cold process route, for example reparative creams/lotions, moisturizing creams/lotions, lightener creams, ointment creams, exfoliating creams, eye shadow creams, color foundation creams or tanner creams (Examples 3-15).

### Example 3

Reparative Cream

| Ingredients | % Weight |
|---|---|
| Octyl Methoxycinnamate | 8.00 |
| Water | 78.60 |
| Safflower Oleosomes (70%) | 7.00 |
| L-Carnitine | 1.00 |
| Decaglycerol L DE (Natrulon ® H 10) | 3.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes along with the L-carnitine and mix at 500 min⁻¹ for 10 min. Add the decaglycerol to the mixture with stirring for 2 min at 500 min⁻¹. Add the methoxycinnamate and the preservative to the mixture and stir at 500 min⁻¹ for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 17,800 mPa·s. The formulation passed stability testing at 45° C. for two months.

### Example 4

Moisturizing Cream

| Ingredients | % Weight |
|---|---|
| Octyl Methoxycinnamate | 8.00 |
| Water | 69.60 |
| Safflower Oleosomes (70%) | 10.00 |
| Urea | 5.00 |
| Decaglycerol L DE (Natrulon ® H 10) | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes with urea and mix at 500 min⁻¹ for 10 min. Add the decaglycerol to the mixture with stirring for 2 min at 500 min⁻¹. Add the methoxycinnamate and the preservative to the mixture and stir at 500 min⁻¹ for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 17,800 mPa·s. The formulation passed stability testing at 45° C. for two months.

### Example 5

Lightener Cream

| Ingredients | % Weight |
|---|---|
| Water | 84.00 |
| Safflower Oleosomes (70%) | 7.00 |
| Arbutin | 1.00 |
| Decaglycerol L DE (Natrulon ® H 10) | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes with arbutin and mix at 500 min⁻¹ for 10 min. Add the decaglycerol to the mixture with stirring for 2 min at 500 min⁻¹. Add the preservative to the mixture and stir at 500 min⁻¹ for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 13,220 mPa·s. The formulation passed stability testing at 45° C. for two months.

### Example 6

Ointment Cream with Vitamin A and E

| Ingredients | % Weight |
|---|---|
| Water | 84.00 |
| Safflower Oleosomes (70%) | 10.00 |
| Vitamin A | 0.5 |
| Vitamin E | 0.5 |
| Decaglycerol L DE (Natrulon ® H 10) | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes with vitamin A and E and mix at 500 min⁻¹ for 10 min. Add the decaglycerol to the mixture with stirring for 2 min at 500 min⁻¹. Add the preservative to the mixture and stir at 500 min⁻for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 12,320 mPa·s. The formulation passed stability testing at 45° C. for two months.

### Example 7

Botanical Exfoliating Cream

| Ingredients | % Weight |
|---|---|
| Water | 72.60 |
| Safflower Oleosomes (70%) | 10.00 |
| Enzyme concentrate¹⁾ | 2.00 |
| Decaglycerol L DE (Natrulon ® H 10) | 5.00 |
| Novemer ® EC-1 | 2.00 |
| Geogard ® 361 (Preservative) | 0.40 |
| Octyl Methoxycinnamate | 8.00 |
| Almond Fragrance # 901A79 | q.s. |

¹⁾18.9 mg papain (62000 PU) + 4.5 mg bromelain (3300 GDU) per 2 g solution (solvent: 50/50 mixture of decaglycerol and water)

### Procedure

Add the water to the safflower oleosomes with papain and bromelain and mix at 500 min⁻¹ for 10 min. Add the decaglycerol to the mixture with string for 2 min at 500 min⁻¹. Add the methoxycinnamate and the preservative to the mixture and stir at 500 min⁻¹ for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min. viscosity 17,310 mPa·s. The formulation passed stability testing at 45° C. for two months.

### Example 8

Eye Shadow Cream

| Ingredients | % Weight |
|---|---|
| Water | 80.30 |
| Safflower Oleosomes (70%) | 12.00 |
| Green Color Pigment A2-2502 | 0.30 |
| Decaglycerol L DE (Natrulon ® H 10) | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes with green color pigment and mix at 500 min⁻¹ for 10 min. Add the decaglycerol to the mixture with stirring for 2 min at 500 min⁻¹. Add the preservative to the mixture and stir at 500 min⁻for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 17,820 mPa·s. The formulation passed stability testing at 45° C. for two months.

### Example 9

Color Foundation Cream

| Ingredients | % Weight |
|---|---|
| Water | 80.10 |
| Safflower Oleosomes (70%) | 12.00 |
| Red Cosmetic Iron Oxide | 0.50 |
| Decaglycerol L DE (Natrulon ® H 10) | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes with red iron oxide color pigment and mix at 500 min⁻¹ for 10 min. Add the decaglycerol to the mixture with stirring for 2 min at 500 min⁻¹. Add the preservative to the mixture and stir at 500 min⁻¹ for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 17,200 mPa·s. The formulation passed stability testing at 45° C. for two months.

### Example 10

Color Foundation/Tanner Cream

| Ingredients | % Weight |
|---|---|
| Water | 80.10 |
| Safflower Oleosomes (70%) | 12.00 |
| Brown Iron Oxide # E 172 | 0.50 |
| Decaglycerol L DE (Natrulon ® H 10) | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes with brown iron oxide color pigment and mix at 500 min⁻¹ for 10 min. Add the decaglycerol to the mixture with stirring for 2 min at 500 min⁻¹. Add the preservative to the mixture and stir at 500 min⁻¹ for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 16,200 mPa·s. The formulation passed stability testing at 45° C. for two months.

### Example 11

Color Blush/Eye Shadow Cream

| Ingredients | % Weight |
|---|---|
| Water | 82.35 |
| Safflower Oleosomes (70%) | 10.00 |
| Red Cosmetic Iron Oxide | 0.25 |
| Decaglycerol L DE (Natrulon ® H 10) | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes with red cosmetic iron oxide color pigment and mix at 500 min⁻¹ for 10 min. Add the decaglycerol to the mixture with stirring for 2 min at 500 min⁻¹. Add the preservative to the mixture and stir at 500 min⁻¹ for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 16,890 mPa·s. The formulation passed stability testing at 45° C. for two months.

### Example 12

Color Blush/Eye Shadow Cream

| Ingredients | % Weight |
|---|---|
| Water | 77.60 |
| Safflower Oleosomes (70%) | 10.00 |
| Red Cosmetic Iron Oxide | 0.25 |
| Decaglycerol L DE (Natrulon ® H 10) | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes with red cosmetic iron oxide color pigment and mix at 500 min⁻¹ for 10 min. Add the decaglycerol to the mixture with stirring for 2 min at 500 min⁻¹. Add the preservative to the mixture and stir at 500 min⁻¹ for 8 min. Add the Novemer® EC-1 and stir at 400 min⁻¹ until the mixture begins to thicken and then increase the stirrer speed to 600 min⁻¹ and stir for an additional 3 min. Total preparation time is ˜23 min, viscosity 16,890 mPa·s. The formulation passed stability testing at 45° C. for two months.

### Example 13

Emulsifier Free Lightener/Exfoliating Cream

| Ingredients | % Weight |
|---|---|
| Water | 77.60 |
| Safflower Oleosomes (70%) | 10.00 |
| Urea | 1.00 |
| Glycerol | 2.00 |
| Rumex occidentalis Extract | 1.00 |
| Kelzan ®-T | 3.00 |
| Titanium Dioxide (Whittaker, Clark & Daniels # 3328) | 5.00 |
| Geogard ® 361 (Preservative) | 0.40 |
| Almond Fragrance # 901A79 | q.s. |

### Procedure

Add the water to the safflower oleosomes with urea and mix at 500 min⁻¹ for 10 min. Add the glycerol and titanium dioxide and Rumex occidentalis extract with stirring for 2 min at 500 min⁻. Add the preservative to the mixture and stir at 500 min⁻¹ for 8 min. Add the Kelzan®-T and stir at 400 min⁻¹ until the mixture begins to thicken and then increase the stirrer speed to 600 min⁻¹ and stir for an additional 3 min. Total preparation time is ˜23 min, viscosity 14,500 mPa·s. The formulation passed stability testing at 45° C. for two months.

### Example 14

Reparative Cream For Dry Skin

| Ingredients | % Weight |
|---|---|
| Octyl Methoxycinnamate | 8.00 |
| Water | 73.60 |
| Safflower Oleosomes (70%) | 10.00 |
| L-Carnitine | 1.00 |
| Decaglyceryl decaoleate | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes along with the L-carnitine and mix at 500 min⁻¹ for 10 min. Add the decaglycerol decaoleate to the methoxycinnamate and the preservative to the mixture and stir at 500 min⁻¹ for 8 min. Add the Novemer® EC-1 and stir at 400 min⁻¹ (5 min at 500 min⁻¹) until the mixture begins to thicken and then increase the stirrer speed to 600 min⁻¹ and stir for an additional 3 min. Total preparation time is ˜26 min, viscosity 17,800 mPa·s. The formulation passed stability testing at 45° C. for two months.

### Example 15

Moisturizing Cream For Dry Skin

| Ingredients | % Weight |
|---|---|
| Octyl Methoxycinnamate | 8.00 |
| Water | 69.60 |
| Safflower Oleosomes (70%) | 10.00 |
| Urea | 5.00 |
| Decaglyceryl decaoleate | 5.00 |
| Novemer ® EC-1 | 2.00 |
| Geogard ® 361 (Preservative) | 0.40 |
| Almond Fragrance # 901A79 | q.s. |

### Procedure

Add the water to the safflower oleosomes with urea and mix at 500 min⁻¹ for 10 min. Add the decaglyceryl decaoleate to the methoxycinnamate and the preservative to the mixture and stir at 500 min⁻¹ for 10 min. Add the Novemer® EC-1 and stir at 400 min⁻¹ (2 min) until the mixture begins to thicken and then increase the stirrer speed to 600 min⁻¹ and stir for an additional 3 min. Total preparation time is ˜25 min, viscosity 17,800 mPa·s. The formulation passed stability testing at 45° C. for two months.

1. A method for preparing a cosmetic or topical dermatological product comprising an oil-in-water emulsion containing at least 60% by weight of water, the method comprising the step of mixing at least one cosmetically or dermatologically active ingredient with an aqueous emulsion of washed vegetable oil bodies (oleosomes), wherein the temperature of said ingredients and said emulsion does never exceed about 40° C.

2. The method of claim 1, wherein the product is a cosmetic product is selected from the group consisting of moisturizers, ointments, cleansers, makeup removers, toners, night treatments, skin reparatives, sunscreens, skin lighteners, color foundations, eye shadows and tanners.

3. The method of claim 1 or 2, wherein the product is a lotion.

4. The method of claim 1 or 2, wherein the product is a cream.

5. The method of any of claims 1 to 4, wherein the vegetable oil bodies (oleosomes) are selected from the group consisting of oil bodies obtained from rapeseed, soybean, sunflower, oil palm, cottonseed, peanut, walnut, coconut, castor, safflower, mustard, coriander, squash, linseed, brazil nut, jojoba, avocado and maize.

6. The method of claim 5, wherein the vegetable oil bodies (oleosomes) are obtained from safflower.

7. The method of any of claims 1 to 6, wherein no additional emulsifier is used.

8. The method of any of claims 1 to 7, wherein the vegetable oil bodies (oleosomes) are used in an amount of from 2 wt. % to 15 wt. % of a 70-75 wt. % aqueous emulsion, based on the total weight of the cosmetic or topical dermatological product.
